(19) United States
(12) Patent Application Publication
(10) Pub. No.: US 2006/0136011 A1
(43) Pub. Date: Jun. 22, 2006

(54) Electronic healthcare device

(76) Inventor: Paul Vo Quang Dang, Salses Le Chateau (FR)

Correspondence Address: HDSL, 4331 STEVENS BATTLE LANE, FAIRFAX, VA, 22033, US

(21) Appl. No.: 11/014,652

(22) Filed: Dec. 17, 2004

(51) Int. Cl. A61N 1/18

(52) U.S. Cl. 607/58

(57) ABSTRACT

An electronic healthcare device includes a microprocessor, a pulse generator and a patch. The microprocessor controls the pulse generator for generating pulses of specific waveforms. An external switch turns on and off the pulse generator and controls the amplitude of the output pulses. The output pulses are sent to two electrodes on the patch via a conducting wire. By attaching the patch to different positions of a human body, one may experience different healthcare effects such as reducing pressure and relaxing muscles, or eradicating smoking addictions.

### BACKGROUND OF THE INVENTION

The present invention relates generally to an electronic healthcare device, and more particularly to an electronic healthcare device including a patch that can generate specific waveforms, whereby the electronic patch attaching to different points of a human body result in different healthcare effects.

In the modem society, more and more people live in an environment full of tension and anxiety, which causes severe health problems. These problems usually come from excessive pressure. Such pressure not only ruins our daily life, but also spoils our performance at work.

However, different people solve such pressure problems in a different manner. Some people try meditation, while others prefer yoga, massage, jogging, walking, or going for a vacation, for example. Although these methods can somehow reduce pressure, they don't always work.

Since different things cause different kinds of pressure and all these pressure accumulate if not properly released. Therefore, when people have no time doing meditation, practicing yoga, doing massage, jogging, walking, or going for a vacation, many people choose smoking or taking a drug for temporarily release or reduce their pressure. Not only pressure without being released, but also victimizing the health.

### BRIEF SUMMARY OF THE INVENTION

The present invention is to solve the drawbacks mentioned above. The electronic healthcare device of the present invention is light in weight and small in size, which is highly portable and extremely convenient for bringing outdoors. One can use the electronic healthcare device of the present invention to reduce pressure and relax muscles at his convenience. It also effectively eradicates smoking addictions, so as to help people to quit smoking completely.

The electronic healthcare device of the present invention includes a microprocessor, a pulse generator and a patch, wherein the microprocessor controls the pulse generator for generating pulses of specific waveforms. An external switch turns on and off the pulse generator and controls the amplitude of the output pulses. The output pulses are sent to two electrodes on the patch via a conducting wire. By attaching the patch to different positions of a human body, one may experience different healthcare effects.

### BRIEF DESCRIPTION OF THE DRAWINGS

FIG. 1 illustrates a perspective view of an electronic healthcare device in accordance with the first embodiment of the present invention.

FIG. 2 illustrates a perspective view of the electronic healthcare device in accordance with the second embodiment of the present invention.

FIG. 3 is a block diagram of the electronic healthcare device of the present invention.

FIG. 4 is a flow diagram illustrating the operation of the electronic healthcare device of the present invention.

FIG. 5 is a table illustrating the waveform sequence of the present invention.

FIG. 6 and FIG. 7 schematically illustrate the electronic healthcare device of the present invention used for reducing pressure.

FIG. 8 and FIG. 9 schematically illustrate the electronic healthcare device of the present invention used for quitting smoking.

### DETAILED DESCRIPTION OF THE INVENTION

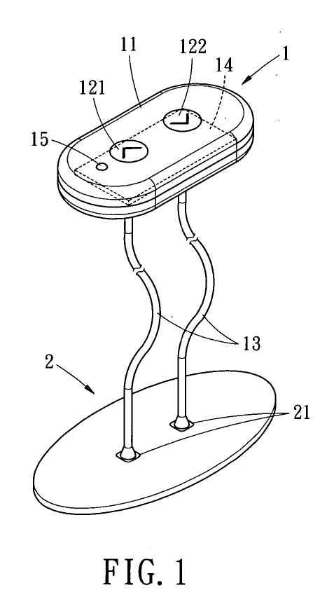

Referring to FIG. 1, a perspective view of an electronic healthcare device of the present invention is illustrated. As shown, the electronic healthcare device includes an electronic patch that is used for attaching to certain positions of human body. The electronic patch employs a series of output pulses for stimulating the positions of human body, so as to effectively achieve the purpose of human body relaxation and to quit smoking.

In light of the above, the electronic healthcare device, in accordance with the first embodiment of the present invention, includes a controller 1 and a patch 2. The controller 1 includes a housing 11 and a circuit board 14 disposed in the housing 11. The housing 11 includes a first button switch 121 and a second button switch 122. The first button switch 121 powers on the electronic healthcare device and increase the stimulation. The functions of powering on and increasing stimulation are integrated in the first button switch 121. When the power of the electronic healthcare device is off, the first button switch 121 acts as a power-on switch. The first button switch 121 becomes a stimulation increment switch when the electronic healthcare device is powered on. On the other hand, the second button switch 122 powers off the electronic healthcare device and decrease the stimulation. The functions of powering off and decreasing stimulation are integrated in the second button switch 121. When the power of the electronic healthcare device is on, the second button switch 122 acts as a power decrement switch. By continuously pressing the second button switch 122, the electronic healthcare device is finally powered off.

A light emitter 15 is configured on the surface of the housing 11, which notifies the user of the electronic healthcare device whether the device is on or off. The circuit board 14 sends the pulses to two electrodes 21 through two conducting wires 13. The two electrodes 21 are formed on one surface of the patch 2, keeping with each other a proper distance. The other surface of the patch 2 is adhesive, which can adhere onto human skin.

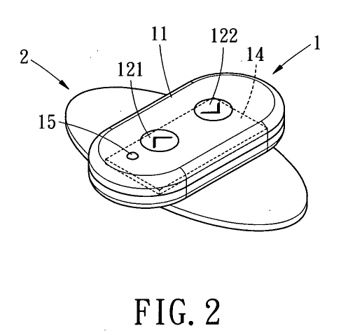

The controller 1 and the patch 2 are separable. As shown in FIG. 1, the controller 1 and the patch 2 are connected with each other through the conducting wires 13. The user can hold the controller 1 in hand for controlling the electronic healthcare device. However, in another embodiment of the present invention, the controller 1 and the patch 2 are combined together without the conductive wires 13, as shown in FIG. 2.

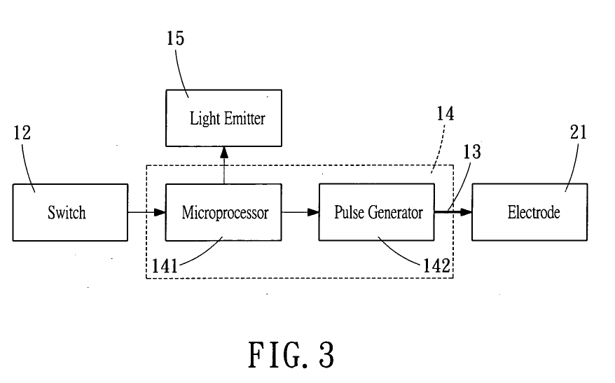

Referring to FIG. 3, a block diagram of the electronic healthcare device of the present invention is illustrated. As shown, the circuit board 14 includes a microprocessor 141 and a pulse generator 142. The pulse generator 142 generates specific pulses under the control of the microprocessor 141. The pulses are then sent to the electrodes 21 via the conducting wires 13. The switch 12 and the light emitter 15 are coupled to the microprocessor 141. The user can input control signals to the microprocessor 141 through the switch 12. The microprocessor 141 then react in accordance with the control signal. The microprocessor also controls the light emitter 15, indicating the electronic healthcare device in either an on state or an off state.

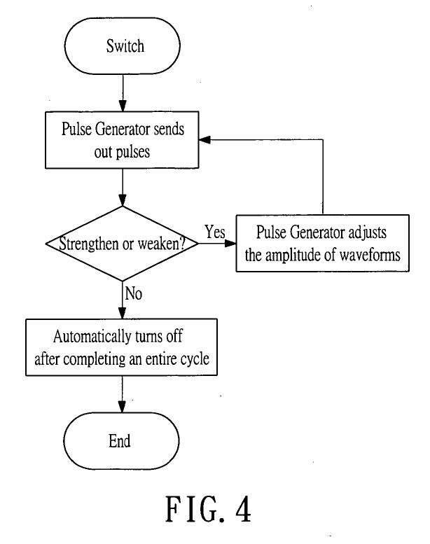

Referring to FIG. 4, a flow diagram showing the operation of the electronic healthcare device of the present invention is illustrated. As shown, after powered on, the pulse generator 142 sends out a series of pulses of specific waveforms. If the user adjusts the strength of the pulses, the pulse generator 142 then adjusts the amplitude of the pulses in accordance with the control of the microprocessor 141 corresponding to user's control. After completing an entire cycle of the waveforms, the microprocessor 141 automatically turns off the pulse generator 142 and stops sending out pulses until the user powers on the electronic healthcare device again.

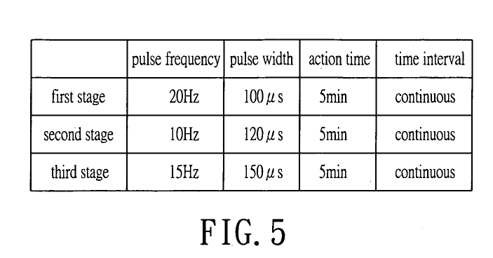

Referring to FIG. 5, the waveform sequence of the present invention is illustrated. As shown, when the pulse generator 42 starts operation, it will sequentially send out waveforms in three different stages. In the first stage, the pulse generator 42 continuously sends out a series of pulses having 20 Hz of frequency and 100 μs of pulse width for an action time of five minutes. In the second stage, the pulse generator 42 continuously sends out a series of pulses having 10 Hz of frequency and 120 μs of pulse width for an action time of five minutes. In the third stage, the pulse generator 42 continuously sends out a series of pulses having 15 Hz of frequency and 150 μs of pulse width for an action time of five minutes. There is a time interval of three seconds between each of the three stages described above. Therefore, one entire cycle is approximately fifteen minutes.

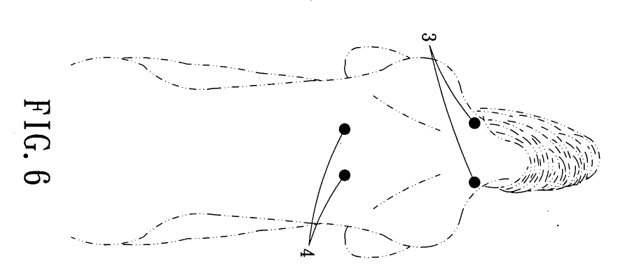

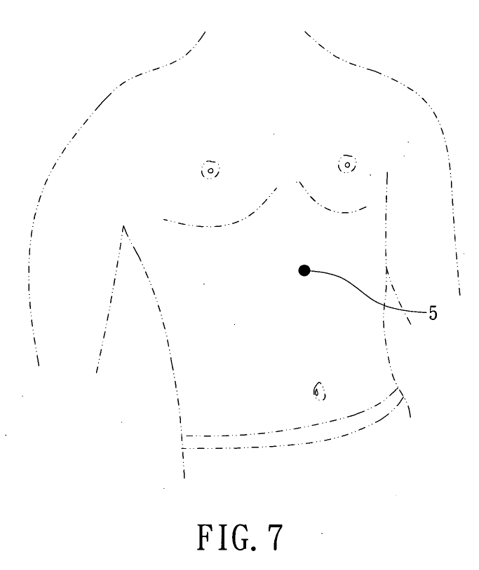

Referring to FIG. 6 and FIG. 7, the electronic healthcare device of the present invention used for reducing pressure is schematically illustrated. As shown, the patch 2 is attached to the first positions 3, the second positions 4 or the third position 5. By stimulating the above positions thirty minutes a day, it can help reducing pressure and relaxing muscles.

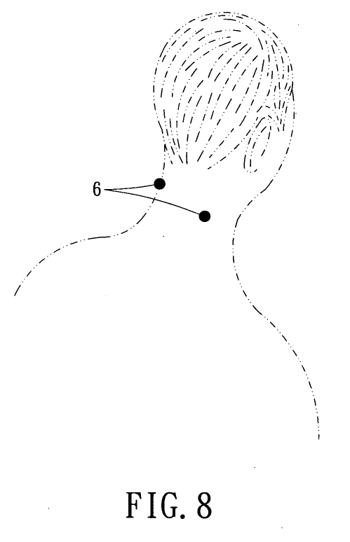

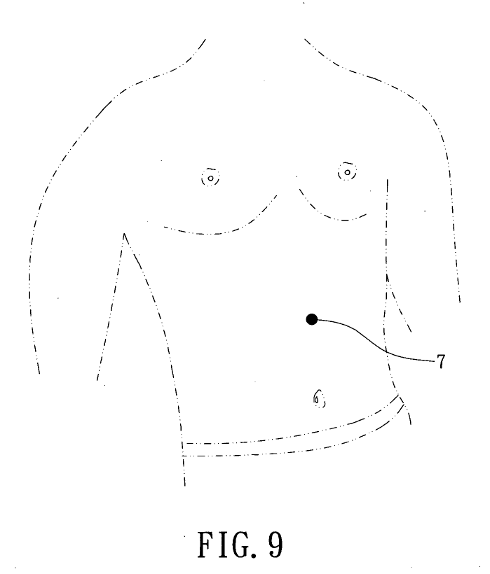

Referring to FIG. 8 and FIG. 9, the electronic healthcare device of the present invention used for quitting smoking is schematically illustrated. As shown, the patch 2 is attached to the first positions 6 or the second position 7. By stimulating the above position thirty minutes a day, it can help not only reducing pressure and relaxing muscles, but also eradicating smoking addiction or quitting smoking.

It is appreciated that the detailed descriptions set forth above are only preferred embodiments of the present invention. Since any person having ordinary skill in the art may readily find various equivalent alterations or modifications in light of the features as disclosed above, it is appreciated that the scope of the present invention is defined in the following claims. Therefore, all such equivalent alterations or modifications without departing from the subject matter as set forth in the following claims is considered within the spirit and scope of the present invention.

What is claimed is:

1. An electronic healthcare device for reducing pressure and eradicating smoking addiction, said electronic healthcare device comprising:
a microprocessor;
a pulse generator controlled under said microprocessor for sending out pulses;
a switch electrically coupled to said microprocessor for inputting control signals;
a patch having an adhesive surface for attaching said patch to human skin;
two electrodes formed on said patch, which is electrically coupled to said pulse generator;
wherein said pulse generator generates pulses in three stages as a cycle, wherein said pulse generator continuously generates pulses having 20 Hz of frequency and 100 μs of pulse width for five minutes in the first stage, pulses having 10 Hz of frequency and 120 μs of pulse width for five minutes in the second stage, and pulses having 15 Hz of frequency and 150 μs of pulse width for five minutes in the third stage.

2. The electronic healthcare device as recited in claim 1, wherein each stage is separated with each other by a time interval of three seconds, making the entire cycle approximately fifteen minutes.

3. The electronic healthcare device as recited in claim 1, further comprising a housing for said microprocessor and said pulse generator disposed inside, and for said switch disposed on one surface thereof.

4. The electronic healthcare device as recited in claim 3, wherein said housing and said patch are integrated with each other.

5. The electronic healthcare device as recited in claim 3, further comprising a conducting wire coupled between said housing and said patch.

6. The electronic healthcare device as recited in claim 1, further comprising a light emitter electrically coupled to said microprocessor for indicating an operation status.

7. The electronic healthcare device as recited in claim 1, wherein said switch comprises a first switch for increasing the amplitude of the pulses and a second switch for decreasing the amplitude of the pulses.

8. The electronic healthcare device as recited in claim 7, wherein said first switch powers on said electronic healthcare device.

9. The electronic healthcare device as recited in claim 7, wherein said second switch powers off said electronic healthcare device.
